(19) United States
(12) Patent Application Publication
(10) Pub. No.: US 2006/0142566 A1
(43) Pub. Date: Jun. 29, 2006

(54) Enantioselective process for the preparation of both enantiomers of 10,11-dihydro-10-hydroxy-5h-dibenz[b,f]azepine-5-carboxamide and new crystal forms thereof

(76) Inventors: Christian Mathes, Offenbury (DE); Gottfried Sedelmeier, Schallstadt (DE); Fritz Blatter, Reinach (CH); Sabine Pfeffer, Weil am Rhein (DE); Dominique Grimler, Hirsingue (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/530,617

(22) PCT Filed: Oct. 6, 2003
(86) PCT No.: PCT/EP03/11034

§ 371 Date: Sep. 9, 2005

(30) Foreign Application Priority Data
Oct. 7, 2002 (GB) 0223224.7

(51) Int. Cl. C07D 223/18

(52) U.S. Cl. 540/590

(57) ABSTRACT

The invention relates to a novel process for the manufacture of substituted enantiopure 10hydroxy-dihydrodibenz[b,f]azepines (Ia), (Ib) wherein each of R¹ and R², independently, are hydrogen, halogen, amino or nitro; and each of R³ and R⁴, independently, are hydrogen or C₁-C₆alkyl; by transfer hydrogenation of 10-oxo-dihydrodibenz[b,f]azepines; and to novel catalysts of formula (III′a) and (III′b) wherein M is Ru, Rh, Ir, Fe, Co or Ni; L₁ is hydrogen; L₂ represents an aryl or aryl-aliphatic residue; and the further radicals have the meanings as defined herein; and to new crystal forms of both enantiomers of 10,11-dihydro-10 hydroxy-5Hdibenz[b,f]azepine-5-carboxamide, obtainable by the new processes, their usage in the production of pharmaceutical preparations, new pharmaceutical preparations comprising these new crystal forms and/or the use of these new crystal forms in the treatment of disorders such as epilepsy, or in the production of pharmaceutical formulations which are suitable for this treatment.

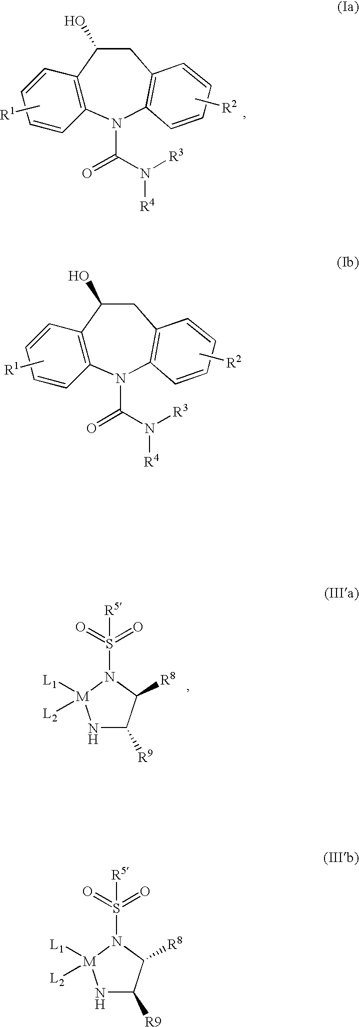

The invention relates to a novel process for the manufacture of substituted enantiopure 10-hydroxy-dihydrodibenz[b,f]azepines by transfer hydrogenation of 10-oxo-dihydrodibenz[b,f]azepines, to novel catalysts and new crystal forms of both enantiomers of 10,11-dihydro-10-hydroxy-5H-dibenz[b,f]azepine-5-carboxamide, obtainable by the new process.

Substituted dihydrodibenz[b,f]azepines are understood to be those active agents which may be preferably used to prevent and treat some central and peripherc nervous system disorders. These compounds are well known and some of them have been used widely for the treatment of some pathological states in humans. For example, 5H-dibenz[b,f]azepine-5-carboxamide (carbamazepine) has become established as an effective agent in the management of epilepsy. An analogue of carbamazepine, 10,11-dihydro-10-oxo-5H-dibenzo[b,f]azepine-5-carbamide (oxcarbazepine, see e.g. German Patent 2.011.087) exhibits comparable antiepileptical activity with less side effects than carbamazepine. Oxcarbazepine is metabolized in mammals to 10,11-dihydro-10-hydroxy-5H-dibenz[b,f]azepine-5-carboxamide (see e.g. Belgian Patent 747.086).

The objective of the present invention is to provide an enantioselective synthesis of substituted 10-hydroxy-dihydrodibenzo[b,f]azepines resulting in high yields and moreover guaranteeing a minimization of the ecological pollution of the environment, being economically attractive, e.g. by using less steps in the reaction and/or process sequence for the manufacture of 10,11-dihydro-10-hydroxy-5H-dibenzo[b,f]azepine-5-carboxamide, and leading to largely enantiomerically pure target products and to products that are possible to crystallize. Furthermore, another objective of the present invention is to provide a process that can be carried out in a larger scale and can thus be used as production process.

Surprisingly, the process of the present invention clearly meets the above objectives.

Accordingly the present invention provides a process for the production of a compound of formula Ia or Ib 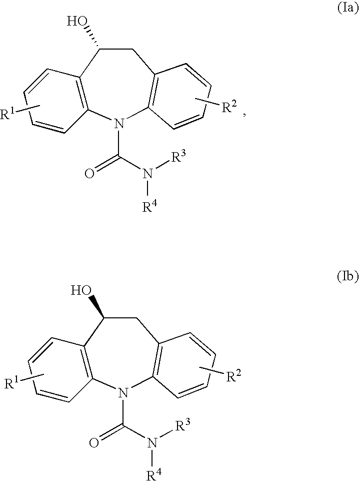
wherein each of R¹ and R², independently, are hydrogen, halogen, amino or nitro; and each of R³ and R⁴, independently, are hydrogen or C₁-C₆alkyl; which process comprises the step of reducing a compound of formula II 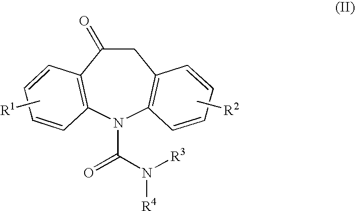
wherein R¹, R², R³ and R⁴ are as defined above; in the presence of a hydrogen donor and a reducing agent selected from the group consisting of a compound of formula (IIIa), (IIIb), (IVa), (IVb), (Va), (Vb), (VIa) or (VIb) 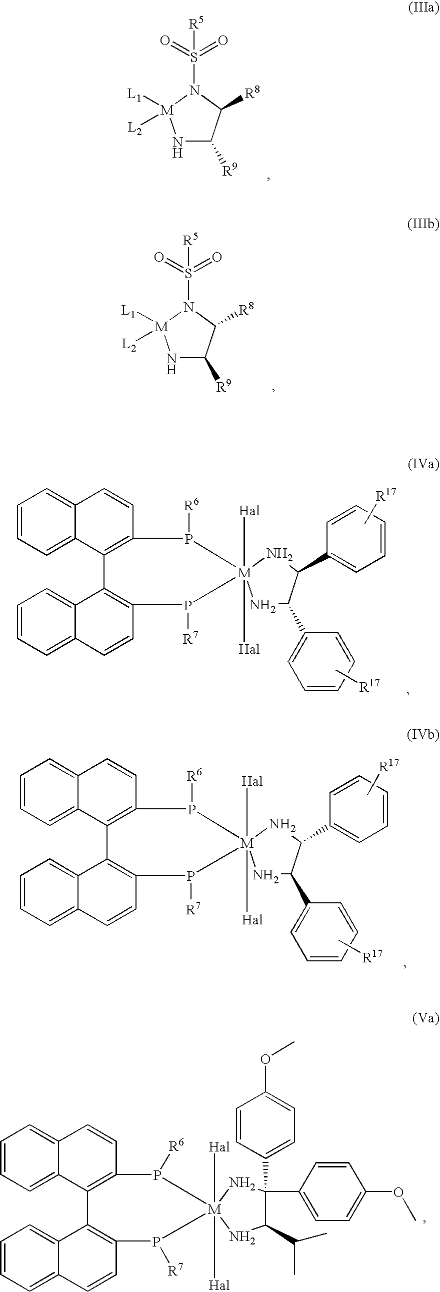 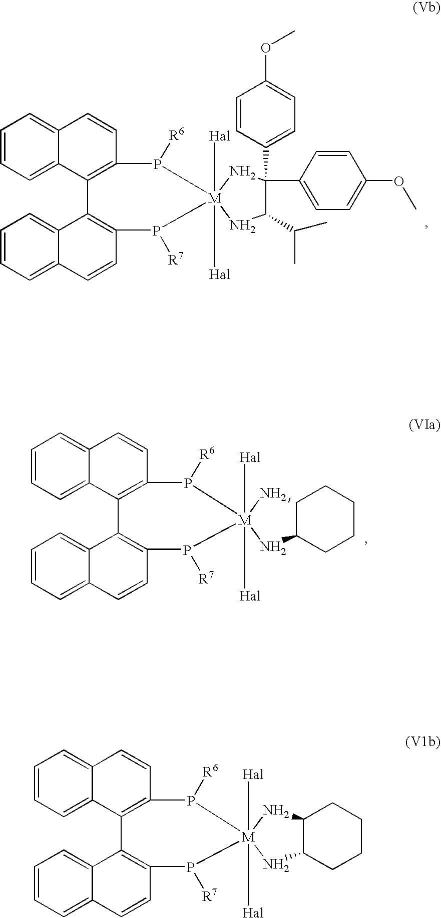
wherein
M is Ru, Rh, Ir, Fe, Co or Ni;
L₁ is hydrogen;
L₂ represents an aryl or aryl-allphatic residue;
Hal is halogen;
R⁵ is an aliphatic, cycloaliphatic, cycloaliphatic-aliphatic, aryl or aryl-aliphatic residue, which, in each case, may be linked to a polymer;
each of R⁶ and R⁷, independently, is an aliphatic, cycloaliphatic, cycloaliphatic-aliphatic, aryl or aryl-aliphatic residue;
each of R⁸ and R⁹ is phenyl or R⁸ and R⁹ form together with the carbon atom to which they are attached a cyclohexane or cyclopentane ring; and
R¹⁷ is H, halogen, amino, nitro or C₁-C₆alkoxy.

For compounds of formula (IVa), (IVb), (Va), (Vb), (VIa) or (VIb), there are combinations with (R)- or (S)-BINAP possible.

Any aromatic residue of a compound of formula (IIIa), (IIIb), (IVa), (IVb), (Va), (Vb), (Via) or (VIb) is substituted or, preferably, unsubstituted. If it is substituted, it may be substituted, for example, by one or more, e.g. two or three, residues e.g. those selected from the group consisting of C₁-C₇alkyl, hydroxy, —O—CH₂—O—, CHO, C₁-C₇alkoxy, C₂-C₈alkanoyl-oxy, halogen, e.g. Cl or F, nitro, cyano, and CF₃.

An aliphatic hydrocarbon residue is, for example, C₁-C₇alkyl, C₂-C₇alkenyl or secondarily C₂-C₇alkynyl. C₂-C₇Alkenyl is in particular C₃-C₇alkenyl and is, for example, 2-propenyl or 1-, 2- or 3-butenyl. C₃-C₅alkenyl is preferred. C₂-C₇Alkynyl is in particular C₃-C₇alkynyl and Is preferably propargyl.

A cycloaliphatic residue is, for example, a C₃-C₈cycloalkyl or, secondarily, C₃-C₈cycloalkenyl. C₃-C₈Cycloalkyl is, for example, cyclopropyl, cyclobutyl, cyclopentyl, cyclohexyl and cycloheptyl. Cyclopentyl and cyclohexyl are preferred. C₃-C₈cycloalkenyl is in particular C₃-C₇cycloalkenyl and is preferably cyclopent-2-en-yl and cyclopent-3-enyl, or cyclohex-2-en-yl and cyclohex-3-en-yl.

A cycloaliphatic-allphatic residue Is, for example, C₃-C₈cycloalkyl-C₁-C₇alkyl, preferably C₃-C₆-cycloalkyl-C₁-C₄alkyl. Preferred is cyclopropylmethyl.

An aryl residue is, for example, a carbocyclic or heterocyclic aromatic residue, in particular phenyl or in particular an appropriate 5- or 6-membered and mono or multicyclic residue which has up to four identical or different hetero atoms, such as nitrogen, oxygen or sulfur atoms, preferably one, two, three or four nitrogen atoms, an oxygen atom or a sulfur atom. Appropriate 5-membered heteroaryl residues are, for example, monoaza-, diaza-, triaza-, tetraaza-, monooxa- or monothia-cyclic aryl radicals, such as pyrrolyl, pyrazolyl, imidazolyl, triazolyl, tetrazolyl, furyl and thienyl, while suitable appropriate 6-membered residues are in particular pyridyl. Appropriate multicyclic residues are anthracenyl, phenanthryl, benzo[1,3]-dioxole or pyrenyl. An aryl residue may be mono-substituted by e.g. NH₂, OH, SO₃H, CHO, or di-substituted by OH or CHO and SO₃H.

An aryl-aliphatic residue is in particular phenyl-C₁-C₇alkyl, also phenyl-C₂-C₇alkenyl or phenyl-C₂-C₇alkynyl.

Halogen represents fluorine, chlorine, bromine or iodine.

Polymers may be polystyrene (PS), cross-linked PS (J), polyethylene glycol (PEG) or a silica gel residue (Si). Examples are NH—R¹⁵ wherein R¹⁵ is C(O)(CH₂)ₙ—PS or C(O)NH(CH₂)ₙ—PS; and —O—Si(R¹⁸)₂(CH₂)ₙR¹⁶ wherein n is 1 to 7, R¹⁸ is C₁-C₆alkyl, e.g. ethyl, and R¹⁶ is a PS, J, PEG or Si (obtainable by Aldrich, Switzerland).

In formula (IIIa), (IIIb), (IVa), (IVb), (Va), (Vb), (VIa) or (VIb) the following significances are preferred independently, collectively or in any combination or sub-combination:

M is Ru, Rh, Ir, preferably Ru.

L₂ is isopropylmethylbenzene, benzene, hexamethylbenzene, mesitylene, preferred is isopropylmethylbenzene.

R⁵ is 2- or 3- or 4-pyridyl, 4-chloro-4-phenoxy-phenyl, 4-phenoxy-phenyl, 5-di(m)ethylamino-1-naphthyl, 5-nitro-1-naphthyl, 2-, 3-, 4-nitrophenyl, 4-vinylphenyl, 4-biphenylyl, 9-anthracenyl, 2-, 3- or 4-hydroxyphenyl, tolyl, phenanthryl, benzo[1,3]-dioxole, dimethyl(naphthalene-1-yl)-amine, trifluoromethyl-phenyl, bis(trifluoromethyl)-phenyl, tris(trifluoromethyl)-phenyl, chrysenyl, perylenyl or pyrenyl.

Each of R⁶ and R⁷, independently, are phenyl, 4-methylphenyl or 3,5-dimethylphenyl, preferred is phenyl.

Each of R⁸ and R⁹ is phenyl or cyclohexyl or substituted phenyl, preferably is phenyl.

Preferred Hal is chloro.

Preferred R¹⁷ is H.

L₁ is as defined above.

A preferred hydrogen donor is, for example, a system comprising 2-propanol, 3-pentanol, or most preferably HOOCH in the presence of an amine, such as triethylamine, DBU or other tertiary amines. The hydrogen donor may also be used as inert solvent, especially 2-propanol and most preferably HCOOH. An alternative hydrogen donor is 2-propanol in the presence of various catalysts and base, e.g. Ru[(1S,2S)-p-TsNCH(C₆H₅)CH(C₆H₅)NH](η⁶-p-cymene) and base or “in situ” [Ru(η⁶-cymene)Cl₂]₂ with chiral ligand (R,R- or S,S-TsDPEN, amino-alcohol) and base. The preferred bases are: t-BuOK, KOH or i-PrOK.

In a preferred aspect, the invention provides a process for the production of a compound of formula I′a or I′b 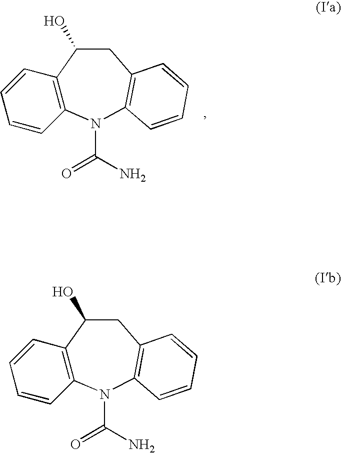
which process comprises the step of reducing the compound of formula II′

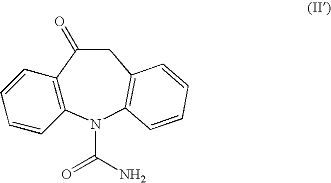

In the presence of a reducing agent selected from the group consisting of a compound of formula (IIIa), (IIIb), (IVa), (IVb), (Va), (Vb), (VIa) or (VIb) as described above and a hydrogen donor.

The compounds of formula II and II′ are known and may be prepared as described In WO-A2-0156992.

The invention further provides the novel compounds of formula III′a and III′b 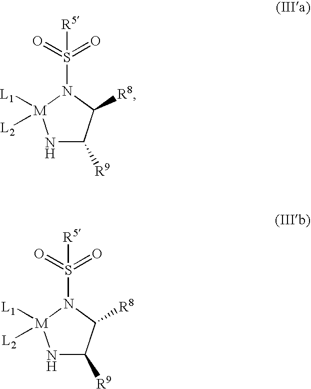
wherein M, L₁, L₂, R⁸ and R⁹ are as defined above and R^{5′} is a group of formula 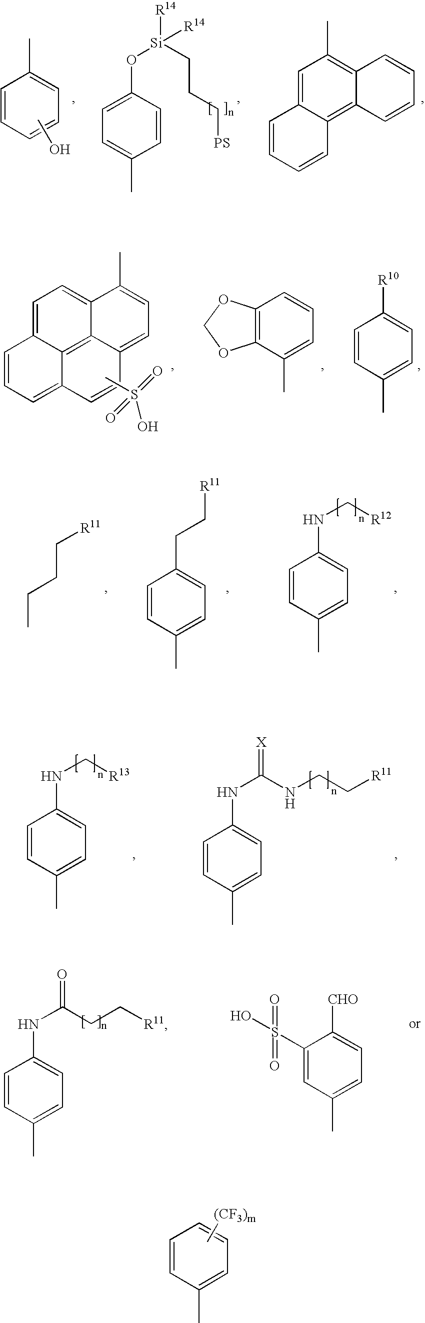
wherein
n is 0, 1, 2, 3, 4, 5, 6 or 7;
X is O or S;
R¹⁰ is polystyrol;
R¹¹ is silica gel;
R¹² is cross-linked polystyrol;
R¹³ is polyethylene-glycol;
R¹⁴ is C₁-C₆alkyl; and
m is 1, 2 or 3.

The following compounds of formula (III′a) or (III′b) wherein L₁, L₂ and R^{5′} are as defined above, are preferred:

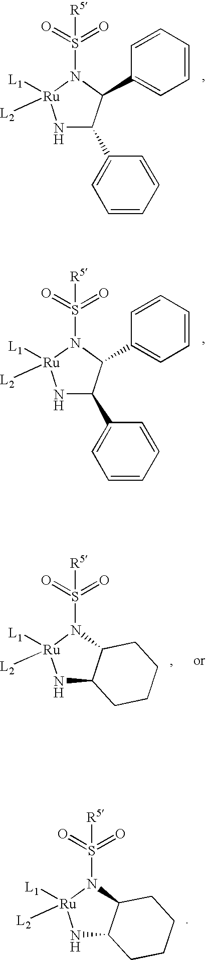

Compounds of formula (III′a) or (III′b) may be prepared by reacting a compound of formula VII 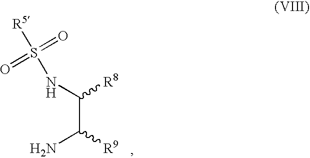
wherein R^{5′}, R⁸ and R⁹ are as defined above, with [MCl₂(p-cymene)]₂ in conventional manner, e.g. as described for M=Ru in the Example 3.

Some compounds of formula (IIIa), (IIIb), (IVa), (IVb), (Va), (Vb), (VIa) or (VIb) are known and may be prepared as described in Haack et al., Angew. Chem., Int. Ed. Engl. 1997, 36, 285-288.

The hydrogenation described above may be carried out, for example in the absence or, customarily, in the presence of a suitable solvent or diluent or a mixture thereof, the reaction, as required, being carried out with cooling, at room temperature or with warming, for example in a temperature range from about −80° C. up to the boiling point of the reaction medium, preferably from about −10° to about +200° C., and, if necessary, in a closed vessel, under pressure, in an inert gas atmosphere and/or under anhydrous conditions.

The hydrogenation may be carried out in a suitable inert solvent, such as an ether, e.g. tetrahydrofuran, an ester, such as ethylacetate, a halogenated solvent, such as methylen-chloride, supercritical CO₂, ionic liquids, a nitrile, especially acetonitrile, an amide, such as dimethylformamide or dimethylacetamide and in a temperature range from, for example, from −78° C., to the boiling point of the solvent, preferably at room temperature, e.g. as described in the Examples.

It is known from the art that asymmetric transfer hydrogenation using a Ru (II) catalyst (esp. a Noyori catalyst) is carried out in the absence of water and under inert gas conditions. Surprisingly, the transfer hydrogenation step according to the present invention can be run in a water containing solvent system and in the absence of an inert gas. This means that the reaction is successful even though the solvent used comprised water (e.g., up to 3% by Karl-Fischer titration).

Optionally, the compounds of formula (I) may be converted into their corresponding pro-drug esters of formula (VIII) 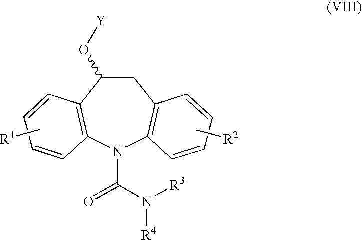
wherein
Y is unbranched or branched C₁-C₁₈alkylcarbonyl, aminoC₁-C₁₈alkylcarbonyl, C₃-C₈cycloalkylcarbonyl, C₃-C₈cycloalkylC₁-C₁₈alkylcarbonyl, halogenC₁-C₁₈alkylcarbonyl, unsubstituted or at the aryl substituted C₅-C₁₀arylC₁-C₁₈alkylcarbonyl, unsubstituted or at the heteroaryl substituted C₅-C₁₀heteroarylC₁-C₁₈alkylcarbonyl, C₁-C₁₈alkoxycarbonyl; and and R¹, R², R³ and R⁴ are as described above (see also EP-B1-751129 for production conditions).

A further objective of the present invention is to provide new crystal forms of both enantiomers of 10,11-dihydro-10hydroxy-5H-dibenz[b,f]azepine-5-carboxamide, obtainable by the new process described above, their usage in the production of pharmaceutical preparations, new pharmaceutical preparations comprising these new crystal forms and/or the use of these new crystal forms in the treatment of disorders such as epilepsy, or in the production of pharmaceutical formulations which are suitable for this treatment.

Hence, the present invention also furnishes new crystal forms of both enantiomers of 10,11-dihydro-10-hydroxy-5H-dibenz[b,f]azepine-5-carboxamide, especially to crystal forms described hereinafter as modification A and modification B.

Neither modification A nor modification B are hygroscopic. Compared to amorphous forms of (S)- or (R)-10,11-dihydro-10-hydroxy-5H-dibenz[b,f]azepine-5-carboxamide, the crystalline forms described herein show a better bulk stability. Furthermore, by the process step of crystallization, the purity of the compounds is increased compared to amorphous material.

Modification A can be distinguished from modification B, for instance, by X-ray powder diffraction techniques, IR spectroscopy and melting points.

The crystal forms can be distinguished in particular by their X-ray powder diffraction pattern. X-ray powder diffraction pattern were taken with a diffractometer and using Cu-Kα₁-radiation are preferably used to characterise solids of organic compounds. X-ray powder diffraction pattern are used particularly successfully to determine the crystal modification of a substance. To characterise the crystal modification A and B of (R)- and (S)-10,11-dihydro-10-hydroxy-5H-dibenz[b,f]azepine-5-carboxamide, respectively, the measurements are made at an angle range (2θ) of e.g. 2° and 45° with samples of substance that are kept at room temperature.

The X-ray powder diffraction pattern thus determined (reflection lines and intensities of the most Important lines) from crystal modification A of (R)-10,11-dihydro-10-hydroxy-5H-dibenz[b,f]azepine-5-carboxamide and (S)-10,11-dihydro-10-hydroxy-5H-dibenz[b,f]azepine-5-carboxamide are both characterised by Table 1.

TABLE 1
Crystal modification A of (R)- or
(S)-10,11-dihydro-10-hydroxy-5H-dibenz[b,f]azepine-S-carboxamide

| Angle | d-spacing | Relative Intensity |
|---|---|---|
| (°2θ) | (Å) | (approximate) |
| 7.0 | 12.6 | m |
| 10.0 | 8.8 | s |
| 11.7 | 7.5 | s |
| 14.1 | 6.28 | vs |
| 16.9 | 5.24 | m |
| 18.0 | 4.93 | m |
| 18.8 | 4.73 | vw |
| 19.4 | 4.58 | w |
| 20.0 | 4.44 | w |
| 20.3 | 4.37 | w |
| 21.8 | 4.08 | w |
| 23.1 | 3.84 | s |
| 23.8 | 3.74 | m |
| 24.2 | 3.67 | w |
| 25.1 | 3.54 | w |
| 25.4 | 3.51 | vw |
| 26.1 | 3.42 | m |
| 26.5 | 3.36 | vw |
| 27.3 | 3.26 | vw |
| 28.6 | 3.12 | w |
| 29.9 | 2.99 | m |
| 31.4 | 2.85 | m |
| 33.0 | 2.71 | w |
| 34.2 | 2.62 | vw |
| 38.2 | 2.35 | w |
| 40.5 | 2.23 | w |
| 44.0 | 2.06 | w |

(vs: very strong, s: strong, m: medium, wv weak, vw: very weak; PXRD was performed on a Philips 1710 powder X-ray diffractometer using Cu_{Kα} radiation. D-spacings were calculated from the 2θ using the wavelength of the Cu_{Kα1} radiation of 1.54060 A. The ratio of Cu_{Kα1} to Cu_{Kα2} radiation was 2:1. The X-ray tube was operated at a Voltage of 40 kV, and a current of 40 mA. A step size of 0.02°, and a counting time of 2.4 s per step was applied. Generally, 2θ values are within an error of ±0.1-0.2°. The experimental error on the d-spacing values is therefore dependent on the peak location.)

The X-ray powder diffraction pattern thus determined (peak positions and intensities of (R)-10,11-dihydro-10-hydroxy-5H-dibenz[b,f]azepine-5-carboxamide and (S)-10,11-dihydro-10-hydroxy-5H-dibenz[b,f]azepine-5-carboxamide are both characterised by Table 2.

TABLE 2
Crystal modification B of (R)- or
(S)-10,11-dihydro-10-hydroxy-5H-dibenz[b,f]azepine-5-carboxamide

| Angle | d-spacing | Relative Intensity |
|---|---|---|
| (°2θ) | (Å) | (qualitative) |
| 9.9 | 8.9 | w |
| 11.4 | 7.8 | s |
| 12.9 | 6.8 | w |
| 14.0 | 6.3 | vs |
| 15.8 | 5.59 | s |
| 17.1 | 5.18 | vw |
| 18.0 | 4.94 | vw |
| 18.9 | 4.69 | w |
| 19.8 | 4.47 | w |
| 20.2 | 4.39 | w |
| 21.5 | 4.13 | m |
| 21.8 | 4.07 | w |
| 22.8 | 3.90 | m |
| 23.6 | 3.76 | s |
| 24.1 | 3.69 | m |
| 25.1 | 3.54 | vw |
| 26.0 | 3.42 | w |
| 26.5 | 3.36 | w |
| 27.1 | 3.29 | w |
| 27.8 | 3.21 | m |
| 29.9 | 2.98 | w |
| 30.8 | 2.90 | w |
| 31.9 | 2.81 | m |
| 34.5 | 2.60 | m |
| 35.5 | 2.53 | w |
| 36.9 | 2.43 | vw |
| 38.4 | 2.34 | vw |
| 44.0 | 2.06 | w |

(vs: very strong, s: strong, m: medium, w: weak, vw: very weak; PXRD was performed on a Philips 1710 powder X-ray diffractometer using Cu_{Kα} radiation. D-spacings were calculated from the 2θ using the wavelength of the Cu_{Kα1} radiation of 1.54060 A. The ratio of Cu_{Kα1} to Cu_{Kα2} radiation was 2:1. The X-ray tube was operated at a Voltage of 40 kV, and a current of 40 mA. A step size of 0.02°, and a counting time of 2.4 s per step was applied. Generally, 2θ values are within an error of ±0.1-0.2°. The experimental error on the d-spacing values is therefore dependent on the peak location.)

Hence, the present invention provides
a crystal form of (R)-10,11-dihydro-10-hydroxy-5H-dibenz[b,f]azepine-5-carboxamide having the reference modification A, which is characterised by a powder X-ray diffraction diagram with d-spacings at 12.6, 8.8, 7.5, 6.28, 5.24, 4.93, 3.84, 3.74 and 3.42 Å, more preferably a crystal form of (R)-10,11-dihydro-10-hydroxy-5H-dibenz[b,f]azepine-5-carboxamide having the reference modification A, which is characterised by a powder X-ray diffraction diagram with d-spacings at 12.6, 8.8, 7.5, 6.28, 5.24, 4.93, 4.58, 4.44, 4.37, 4.08, 3.84, 3.74, 3.67, 3.54, 3.42, 3.12 and 2.71 Å,
a crystal form of (R)-10,11-dihydro-10-hydroxy-5H-dibenz[b,f]azepine-5-carboxamide having the reference modification B, which is characterised by a powder X-ray diffraction diagram with d-spacings at 8.9, 7.8, 6.8, 6.3, 5.59, 4.13, 3.90, 3.69, 3.29 and 2.60 Å, more preferably a crystal form of (R)-10,11-dihydro-10-hydroxy-5H-dibenz[b,f]azepine-5-carboxamide having the reference modification B, which is characterised by a powder X-ray diffraction diagram with d-spacings at 8.9, 7.8, 6.8, 6.3, 5.59, 4.69, 4.47, 4.39, 4.13, 4.07, 3.90, 3.69, 3.42, 3.36, 3.29, 2.98, 2.90 and 2.60 Å,
a crystal form of (S)-10,11-dihydro-10-hydroxy-5H-dibenz[b,f]azepine-5-carboxamide having the reference modification A, which is characterised by a powder X-ray diffraction diagram with d-spacings at 12.6, 8.8, 7.5, 6.28, 5.24, 4.93, 3.84, 3.74 and 3.42 Å, more preferably a crystal form of (R)-10,11-dihydro-10-hydroxy-5H-dibenz[b,f]azepine-5-carboxamide having the reference modification A, which Is characterised by a powder X-ray diffraction diagram with d-spacings at 12.6, 8.8, 7.5, 6.28, 5.24, 4.93, 4.58, 4.44, 4.37, 4.08, 3.84, 3.74, 3.67, 3.54, 3.42, 3.12 and 2.71 Å, and
a crystal form of (S)-10,11-dihydro-10-hydroxy-5H-dibenz[b,f]azepine-5-carboxamide having the reference modification B, which is characterised by a powder X-ray diffraction diagram with d-spacings at 8.9, 7.8, 6.8, 6.3, 5.59, 4.13, 3.90, 3.69, 3.29 and 2.60 Å, more preferably a crystal form of (R)-10,11-dihydro-10-hydroxy-5H-dibenz[b,f]azepine-5-carboxamide having the reference modification B, which is characterised by a powder X-ray diffraction diagram with d-spacings at 8.9, 7.8, 6.8, 6.3, 5.59, 4.69, 4.47, 4.39, 4.13, 4.07, 3.90, 3.69, 3.42, 3.36, 3.29, 2.98, 2.90 and 2.60 Å.

In the infrared spectra, a number of differences between the two crystal modifications can be observed, e.g. a shift of the major carbonyl absorption. For instance, in the IR spectrum of crystal modification B of (S)-10,11-dihydro-10-hydroxy-5H-dibenz[b,f]azepine-5-carboxamide a strong absorption (presumably the carbonyl absorption) is observed between about 1657 to 1659 cm⁻¹, whereas in the IR spectrum of crystal modification A of (S)-10,11-dihydro-10-hydroxy-5H-dibenz[b,f]azepine-5-carboxamide strong absorption is observed between about 1649 to 1651 cm⁻¹. Another strong absorption in the IR spectrum of crystal modification B of (S)-10,11-dihydro-10-hydroxy-5H-dibenz[b,f]azepine-5-carboxamide is observed between about 1584 to 1586 cm⁻¹, whereas in the IR spectrum of crystal modification A of (S)-10,11-dihydro-10-hydroxy-5H-dibenz[b,f]azepine-5-carboxamide this absorption is shifted to values between about 1564 to 1566 cm⁻¹.

Furthermore, it was found that crystal modification B of (S)-10,11-dihydro-10-hydroxy-5H-dibenz[b,f]azepine-5-carboxamide has a melting point between 193.0 and 197.0° C., especially a melting point between 194.0 and 196.0° C., e.g. 195.5° C. Hence the present invention also relates to a crystal modification of (S)-10,11-dihydro-10-hydroxy-5H-dibenz[b,f]azepine-5-carboxamide having a melting point between 193.0 and 197.0° C. especially a melting point between 194.0 and 196.0° C., e.g. 195.5° C.

The invention also relates to a new anhydrous crystal form of (R)- or (S)-10,11-dihydro-10-hydroxy-5H-dibenz[b,f]azepine-5-carboxamide, which is characterised by a melting enthalpy of between 122 J/g and 136 J/g, preferably between 126 and 131 J/g, more preferably between 128 and 129 J/g.

Crystal modification A of (R)- or (S)-10,11-dihydro-10-hydroxy-5H-dibenz[b,f]azepine-5-carboxamide can be obtained by quickly precipitating (R)- or (S)-10,11-dihydro-10-hydroxy-5H-dibenz[b,f]azepine-5-carboxamide, respectively, from its solution in a suitable solvent, e.g. dichloromethane, acetone or an alcohol such as ethanol or isopropanol, e.g. by first warming a saturated solution of (R)- or (S)-10,11-dihydro-10-hydroxy-5H-dibenz[b,f]azepine-5-carboxamide, respectively, to reflux temperature and thereafter allowing crystallization at room temperature.

Crystal modification B of (R)- or (S)-10,11-dihydro-10-hydroxy-5H-dibenz[b,f]azepine-5-carboxamide can be obtained from the corresponding crystal modification A or from amorphous material by phase equilibration in a suitable solvent, e.g. by vibration for 12 to 200 hours, e.g. 24 hours, in acetone or ethanol at room temperature. The time necessary to obtain pure form B depends on the enantiomer and the particular solvent used. For instance, (S)-10,11-dihydro-10-hydroxy-5H-dibenz[b,f]azepine-5-carboxamide having crystal modification A can be transferred into (S)-10,11-dihydro-10-hydroxy-5H-dibenz[b,f]azepine-5-carboxamide having crystal modification B in acetone at room temperature in less than 24 hours.

Furthermore, crystal modification B of (R)- or (S)-10,11-dihydro-10-hydroxy-5H-dibenz[b,f]azepine-5-carboxamide can be obtained by crystallization of (R)- or (S)-10,11-dihydro-10-hydroxy-5H-dibenz[b,f]azepine-5-carboxamide from its solution in a suitable solvent, e.g. an alcohol such as ethanol or isopropanol, especially by adding a crystal of (R)- or (S)-10,11-dihydro-10-hydroxy-5H-dibenz[b,f]azepine-5-carboxamide, respectively, having crystal modification B.

By the procedures described herein, the distinct crystal modifications A and B of the (R)- and (S)-enantiomer, respectively, can be obtained in pure form, i.e. the pure entaniomers are obtained in a crystal form which contains less than 10% of the other crystal form, preferably less than 5% of the other crystal form, more preferably less than 1% of the other crystal form.

Hence the present invention furnishes
a process for the preparation of (R)- or (S)-10,11-dihydro-10-hydroxy-5H-dibenz[b,f]azepine-5-carboxamide having crystal form B, wherein (a) (R)- or (S)-10,11-dihydro-10-hydroxy-5H-dibenz[b,f]azepine-5-carboxamide are prepared according to a process according to any one of claims 2 to 4 for the enantioselective production of a compound of formula I′a or I′b, and (b) the obtained product having crystal modification A or being in from amorphous form, is subjected to phase equilibration in a suitable solvent;

a process for the preparation of (R)- or (S)-10,11-dihydro-10-hydroxy-5H-dibenz[b,f]azepine-5-carboxamide having crystal form B, wherein (R)- or (S)-10,11-dihydro-10-hydroxy-5H-dibenz[b,f]azepine-5-carboxamide are prepared according to a process according to any one of claims 2 to 4 for the enantioselective production of a compound of formula I′a or I′b, and the obtained product having crystal modification A or being in from amorphous form, is solved in a suitable solvent and a crystal of (R)- or (S)-10,11-dihydro-10-hydroxy-5H-dibenz[b,f]azepine-5-carboxamide, respectively, having crystal modification B is added;
a process for the preparation of (R)- or (S)-10,11-dihydro-10-hydroxy-5H-dibenz[b,f]azepine-5-carboxamide having crystal form B, wherein (R)- or (S)-10,11-dihydro-10-hydroxy-5H-dibenz[b,f]azepine-5-carboxamide having crystal modification A or being in an amorphous form, is subjected to phase equilibration or crystallization in a suitable solvent; and
a process for the preparation of (R)- or (S)-10,11-dihydro-10-hydroxy-5H-dibenz[b,f]azepine-5-carboxamide having crystal form B, wherein (R)- or (S)-10,11-dihydro-10-hydroxy-5H-dibenz[b,f]azepine-5-carboxamide having crystal modification A or being in an amorphous form, is solved in a suitable solvent and a crystal of (R)- or (S)-10,11-dihydro-10-hydroxy-5H-dibenz[b,f]azepine-5-carboxamide, respectively, having crystal modification B is added (seeding).
the crystal form of (R)-10,11-dihydro-10-hydroxy-5H-dibenz[b,f]azepine-5-carboxamide having the reference modification B described herein comprising less than 5% of modification A.
the crystal form of (S)-10,11-dihydro-10-hydroxy-5H-dibenz[b,f]azepine-5-carboxamide having the reference modification B described herein comprising less than 5% of modification A.

The new crystal forms are especially stable, in particular crystal form B is to be regarded as the one which is the thermodynamically stable crystalline form, and they are therefore suitable as active ingredients for solid forms of administration, for storing in solid form or as intermediates (with particularly good storability) in the preparation of solid or liquid forms of administration. Upon storage of modification B, no crystals of modification A should be obtained. Such stable forms are preferred for the preparation of medicaments.

On the other hand, modification A is better soluble in organic and aqueous solutions than modification B and, hence, is more suitable for the preparation of infusions. Furthermore, modification A can be incorporated in solid dosage forms such as tablets in order to have an improved, in particular a faster, bioavailability than modification B.

The invention also relates to the use of the new crystal forms in the production of pharmaceutical preparations, new pharmaceutical preparations which contain these new crystal forms, and/or their use in the treatment of epilepsy. In the following, where pharmaceutical preparations or compositions which comprise or contain the active ingredient are mentioned, in the case of liquid compositions or compositions which no longer contain the crystal form as such, this is always understood to mean also the pharmaceutical preparations obtainable using the crystal forms (for example infusion solutions obtained using crystal forms A or B as defined herein), even if they no longer contain the respective crystal form (for example because they exist in solution).

The invention also relates especially to the use of a new crystal form of (R)- or (S)-10,11-dihydro-10-hydroxy-5H-dibenz[b,f]azepine-5-carboxamide having crystal form A or, preferably, B, in the production of pharmaceutical preparations, characterised by mixing a new crystal form of (R)- or (S)-10,11-dihydro-10-hydroxy-5H-dibenz[b,f]azepine-5-carboxamide having crystal form A or B with one or more carriers.

The invention also relates to a method of treating warm-blooded animals suffering from a disorder such as epilepsy, characterised by administering a dose of (R)- or (S)-10,11-dihydro-10-hydroxy-5H-dibenz[b,f]azepine-5-carboxamide which is effective for treating said disease in one of the new crystal forms to a warm-blooded animal requiring such treatment, also including in particular the treatment with those preparations that are produced using one of the new crystal forms; and/or the use of a new crystal form of (R)- or (S)-10,11-dihydro-10-hydroxy-5H-dibenz[b,f]azepine-5-carboxamide having crystal form A or B in such a treatment.

To produce the pharmaceutical preparations, the active ingredient may be used for example in such a way that the pharmaceutical preparations contain an effective amount of the active ingredient together or in a mixture with a significant amount of one or more organic or inorganic, liquid or solid, pharmaceutically acceptable carriers.

The pharmaceutical compositions according to the invention are those intended for enteral, especially nasal, rectal or oral, or parenteral administration to warm-blooded animals, especially humans, and they contain an effective dose of the active ingredient on its own or together with a significant amount of a pharmaceutically acceptable carrier. The dose of the active ingredient is dependent on the type of warm-blooded animal, the body weight, the age and the individual condition, individual pharmacokinetic situations, the disease to be treated and the type of administration.

The following Examples illustrate the invention.

Abbreviations

aqu. Aqueous

dansyl 5-(dimethylamino)-1-naphthalenesulfonyl

ee enantiomeric purity

Et ethyl

EtOAc ethyl acetate

HPLC high pressure liquid chromatography

Me methyl

NMR nuclear magnetic resonance

RT room temperature

THF tetrahydrofuran

Ts tosyl

Differential Scannig Calorimetry (DSC)

DSC investigations are made on a Perkin Elmer DSC 7 instrument or on Perkin Elmer Pyris DSC. About 2-4 mg of drug substance are place into a gold sample pan which is sealed under nitrogen to prevent oxidation during the heating phase. A heating rate of 10° C./min is applied from 25° C. to 210° C.

Powder X-ray Diffraction (PXRD)

PXRD is performed on a Philips 1710 powder X-ray diffractometer using Cu_{Kα} radiation. The X-ray tube is operated at a Voltage of 40 kV, and a current of 40 mA. A step size of 0.02°, and a counting time of 2.4 s per step is applied.

Infrared Spectroscopy (IR)

IR is performed on a Perkin-Elmer BX II FT-IR spectrometer. About 1 mg of drug substance are pressed into a KBr pellet. 12 scans at a resolution of 2 cm⁻¹ are acquired. For characterization of the polymorphs ATR-IR is performed using a Greasby Specac Golden Gate Diamond ATR Accessory, Serial No. 2585. About 10 mg of test substance are pressed In the ATR cell using 70 cNm.

### EXAMPLE 1

### Procedure for the Enantioselective Transfer Hydrogenation of 10-Oxo-10,11-dihydro-dibenzo[b,f]azepine-5-carboxylic acid amide to R(−)-10,11-Dihydro-10-hydroxy-5H-dibenz[b,f]azepine-5-carboxamide

To a mixture of 10-oxo-10,11-dihydro-dibenzo[b,f]azepine-5-carboxylic acid amide (300 mg, 1.189 mmol) and RuCl[(1R,2R)-p-TsNCH(C₆H₅)CH(C₆H₅)NH₂](η⁶-p-cymene, Aldrich, Switzerland) (8.8 mg, 0.0138 mmol) in CH₂Cl₂ (15 ml) is added dropwise a premixed solution of formic acid and NEt₃ (5:2, 328 mg:289 mg) at 23 IC and stirred for 10 min. The clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of R(−)-10,11-dihydro-10-hydroxy-5H-dibenzo[b,f]azepine-5-carboxamide (enantiomeric purity (ee)>99% determined by HPLC on Chiracel OD, Retention time: 9.46 min. [α]_{D}^{rt}=−195.3° (ethanol). ¹H-NMR (400 MHz, CDCl₃):7.70-7.20 (m, 8H), 5.30 (br s, 1H), 5.10-4.60 (br s, 2H), 3.75-3.40 (m, 1H), 3.20-2.90 (m, 1H), 2.50 (br s, 2H). NMR data refer to Lit.: Benes, J et al., J. Med. Chem. 1999, 42, 2582-2587. Molecular weight: 254.291

### EXAMPLE 2

### Procedure for the Enantioselective Transfer Hydrogenation of 10-Oxo-10,11-dihydro-dibenzo[b,f]azepine-5-carboxylic acid amide to S(+)-10,11-Dihydro-10-hydroxy-5H dibenz[b,f]azepine-5-carboxamide

To a mixture of 10-Oxo-10,11-dihydro-dibenzo[b,f]azepine-5-carboxylic acid amide (300 mg, 1.189 mmol) and RuCl[(1S,2S)-p-TsNCH(C₆H₅)CH(C₆H₅)NH₂](η⁶-p-cymene) (11 mg, 0.0173 mmol) in CH₂Cl₂ (15 ml) is added in two portions a premixed solution of formic acid and NEt₃ (5:2, 656 mg:578 mg) at 23° C. and stirred for 10 min. After that formic acid is added (50 μl) and the clear solution is heated to reflux for 16 h. The reaction mixture Is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of S(+)-10,11-dihydro-10-hydroxy-5H-dibenzo[b,f]azepine-5-carboxamide (ee>99% by HPLC on Chiracel OD). Retention time: 12.00 min. [α]_{D}^{rt}=+196.6° (ethanol). ¹H-NMR (400 MHz, CDCl₃):7.70-7.20 (m, 8H), 5.30 (br s, 1H), 5.10-4.60 (br s, 2H), 3.75-3.40 (m, 1H), 3.20-2.90 (m, 1H), 2.50 (br s, 2H). NMR data refer to Lit.: Benes, J et al., J. Med. Chem. 1999, 42, 2582-2587. Molecular weight: 254.291

Alternative production: To a mixture of 10-oxo-10,11-dihydro-dibenzo[b,f]azepine-5-carboxylic acid amide (300 mg, 1.189 mmol) and RuCl[(1S,2S)-p-dansyl-NCH(C₆H₅)CH(C₆H₅)NH₂](η⁶-p-cymene) (8.5 mg, 0.012 mmol) in CH₂Cl₂ (15 ml) is added dropwise a premixed solution of formic acid and NEt₃ (5:2, 328 mg:289 mg) at 23° C. and stirred for 10 min. The clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of S(+)-10,11-Dihydro-10-hydroxy-5H-dibenzo[b,f]azepine-5-carboxamide.

### EXAMPLE 3

### Preparation of RuCl[(1S,2S)-p-dansylNCH(C₆H₅)CH(C₆H₅)NH₂](η⁶-cymene)

a) Preparation of (S,S)-5-dimethylamino-naphthalene-1-sulfonic acid (2-amino-1,2-diphenylethyl)-amide: To a solution of (S,S)-diphenylethylenediamine (250 mg, 1.2 mmol) and triethylamine (0.5 ml) in THF is added dropwise a solution of dansyl chloride (318 mg, 1.2 mmol) in THF (2 ml) at 0° C. After stirring 16 h at RT the solvent is removed in vacuum and the residue is resolved in methylenehloride (20 ml). The organic solution is washed with NaHCO₃ solution (5 ml), dried over Na₂SO₄ and after filtration the solvent is removed. Flash chromatographie afford (S,S)-5-dimethylamino-naphthalene-1-sulfonic acid (2-amino-1,2-diphenyl-ethyl)-amide as yellow oil which crystallizes by drying in vacuum. M: 445.59. ¹H-NMR (400 MHz, CDCl₃):8.36 (t, J=7.5 Hz, 2H), 8.17 (dd, J=7.2, 1.2 Hz, 1H), 7.47 (dd, J=8.8 Hz, 1H), 7.34 (dd, J=8.5 Hz, 1H), 7.24-7.16 (m, 4H), 7.11 (d, J=7.5 Hz, 1H), 6.99-6.74 (m, 6H), 4.61 (d, J=8.5 Hz, 1H), 4.20 (d, J=8.5 Hz, 1H), 2.80 (s, 6H).

b) Preparation of RuCl[(1S,2S)-p-dansylNCH(C₆H₅)CH(C₆H₅)NH₂](η⁶-p-cymene): A solution of (S,S)-5-dimethylamino-naphthalene-1-sulfonic acid (2-amino-1,2-diphenyl-ethyl)-amide (80 mg, 0.18 mmol), NEt₃ (36 mg, 0.36 mmol) and [RuCl₂(p-cymene)]₂ (55 mg, 0.09 mmol) in 2-propanol is heated at 80° C. for 1 h. The solvent is removed after that und the dark red residue is washed with water (2 ml). The solid is dried in vacuum and used without any purification. M: 715.34.

### EXAMPLE 4

### Crystal Modification B of (R)-10,11-Dihydro-10-hydroxy-5H-dibenz[b,f]azepine-5-carboxamide

120 mg of crystal modification A of (R)-10,11-dihydro-10-hydroxy-5H-dibenz[b,f]azepine-5-carboxamide are suspended in 1.0 ml of acetone and the obtained suspension is stirred with a magnetic stirrer shaken for 160 hours at 21 to 25° C. The product is filtered and dried in air at room temperature providing crystal modification B of (R)-10,11-dihydro-10-hydroxy-5H-dibenz[b,f]azepine-5-carboxamide in the form of white crystals.

### EXAMPLE 5

### Crystal Modification B of (S)-10,11-Dihydro-10-hydroxy-5H-dibenz[b,f]azepine-5-carboxamide

120 mg of crystal modification A of (S)-10,11-dihydro-10-hydroxy-5H-dibenz[b,f]azepine-5-carboxamide are suspended in 1.0 ml of acetone and the obtained suspension is stirred with a magnetic stirrer shaken for 24 hours at 21 to 25° C. The product is filtered and dried in air at room temperature providing crystal modification B of (S)-10,11-dihydro-10-hydroxy-5H-dibenz[b,f]azepine-5-carboxamide in the form of white crystals.

1. A process for the production of a compound of formula Ia or Ib 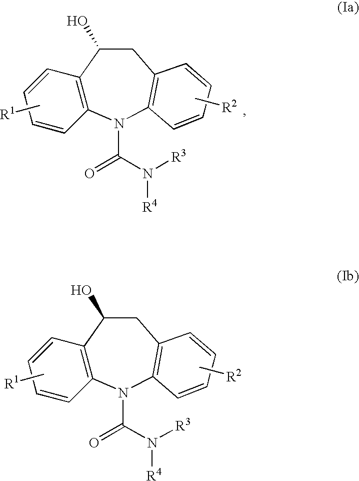
wherein
each of R¹ and R², independently, are hydrogen, halogen, amino or nitro; and
each of R³ and R⁴, independently, are hydrogen or C₁-C₆alkyl;
which process comprises the step of reducing a compound of formula II 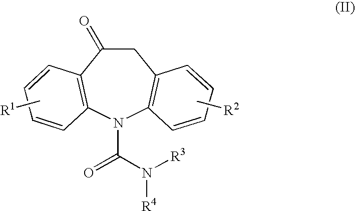
wherein R¹, R², R³ and R⁴ are as defined for a compound of formula Ia or Ib; in the presence of a hydrogen donor and a reducing agent selected from the group consisting of the compounds of formula (IIIa), (IIIb), (IVa), (IVb), (Va), (Vb), (VIa) or (VIb) 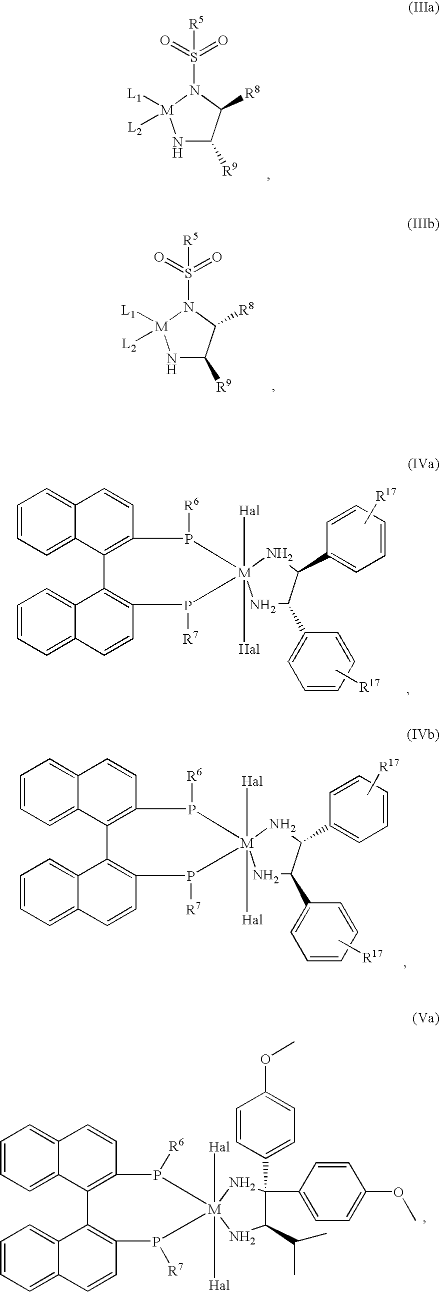 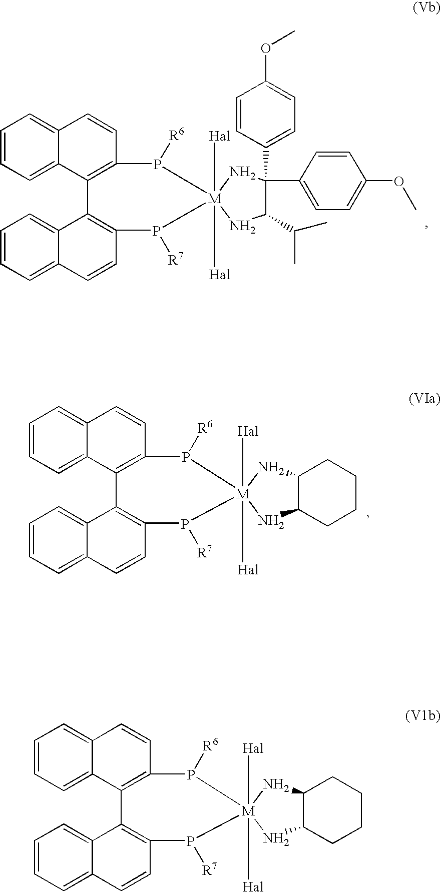
wherein
M is Ru, Rh, Ir, Fe, Co or Ni;
L₁ is hydrogen;
L₂ represents an aryl or aryl-aliphatic residue;
Hal is halogen;
R⁵ is an aliphatic, cycloaliphatic, cycloaliphatic-aliphatic, aryl or aryl-aliphatic residue, which, in each case, may be linked to a polymer;
each of R⁶ and R⁷, independently, is an aliphatic, cycloaliphatic, cycloaliphatic-aliphatic, aryl or aryl-aliphatic residue;
each of R⁸ and R⁹ is phenyl or R⁸ and R⁹ form together with the carbon atom to which they are attached a cyclohexyen or cyclopenten ring; and
R¹⁷ is H, alkyl, halogen, amino, dialkylamino, nitro or C₁-C₆alkoxy.

2. The process according to claim 1 for the production of a compound of formula I′a or I′b

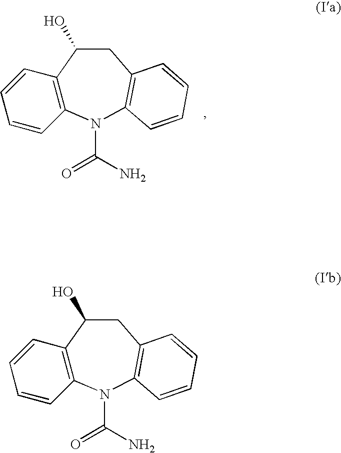

3. The process according to claim 1 wherein the transfer hydrogenation step takes place in a water containing solvent system.

4. The process according to claim 3 wherein the transfer hydrogenation step takes place in the absence of an inert gas.

5. A compound of formula III′a and III′b 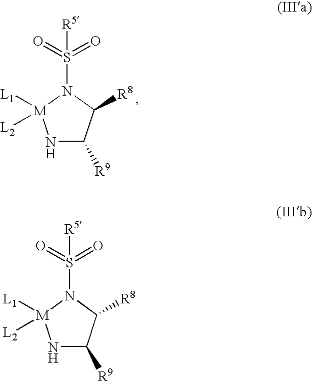
wherein
M is Ru, Rh, Ir, Fe, Co or Ni;
L₁ is hydrogen;
L₂ represents an aryl or aryl-aliphatic residue;
each of R⁸ and R⁹ is phenyl or R⁸ and R⁹ form together with the carbon atom to which they are attached a cyclohexyen or cyclopenten ring; and
R^{5′} is a group of formula 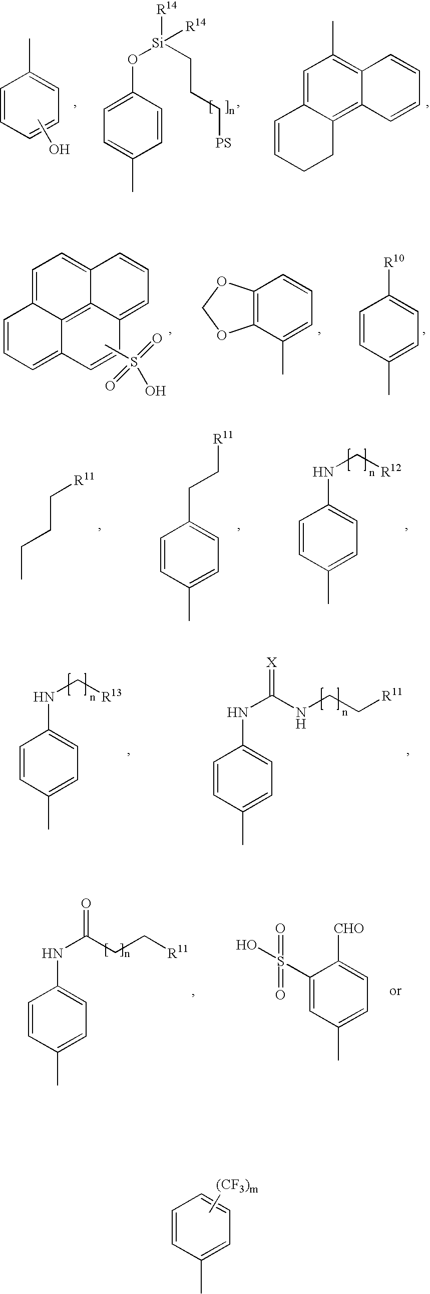
wherein
n is 0, 1, 2, 3, 4, 5, 6 or 7;
X is O or S;
R¹⁰ is polystyrol;
R¹¹ is silica gel;
R¹² is cross-linked polystyrol;
R¹³ is polyethylene-glycol;
R¹⁴ is C₁-C₆alkyl; and
m is 1, 2 or 3;
or a salt thereof.

6. A crystal form of (R)-10,11-dihydro-10-hydroxy-5H-dibenz[b,f]azepine-5 carboxamide having the reference modification A, which is characterised by a powder X-ray diffraction diagram with d-spacings at 12.6, 8.8, 7.5, 6.28, 5.24, 4.93, 3.84, 3.74, 3.42 Å.

7. A crystal form of (R)-10,11-dihydro-10-hydroxy-5H-dibenz[b,f]azepine-5 carboxamide having the reference modification B, which is characterised by a powder X-ray diffraction diagram with d-spacings at 8.9, 7.8, 6.8, 6.3, 5.59, 4.13, 3.90, 3.69, 3.29, 2.60 Å.

8. A crystal form of (S)-10,11-dihydro-10-hydroxy-5H-dibenz[b,f]azepine-5 carboxamide having the reference modification A, which is characterised by a powder X-ray diffraction diagram with d-spacings at 12.6, 8.8, 7.5, 6.28, 5.24, 4.93, 3.84, 3.74, 3.42 Å.

9. A crystal form of (S)-10,11-dihydro-10-hydroxy-5H-dibenz[b,f]azepine-5 carboxamide having the reference modification B, which is characterised by a powder X-ray diffraction diagram with d-spacings at 8.9, 7.8, 6.8, 6.3, 5.59, 4.13, 3.90, 3.69, 3.29, 2.60 Å.

10. An anhydrous crystal form of (R)- or (S)-10,11-dihydro-10-hydroxy-5H dibenz[b,f]azepine-5-carboxamide, which is characterised by a melting enthalpy of between 122 J/g and 136 J/g.

11. The crystal form of (R)-10,11-dihydro-10-hydroxy-5H dibenz[b,f]azepine-5 carboxamide having the reference modification B, which is characterised by a powder X-ray diffraction diagram with d-spacings at 8.9, 7.8, 6.8, 6.3, 5.59, 4.13, 3.90, 3.69, 3.29, 2.60 Å and comprising less than 5% of reference modification A, which is characterised by a powder X-ray diffraction diagram with d-spacings at 12.6, 8.8, 7.5, 6.28, 5.24, 4.93, 3.84, 3.74, 3.42 Å.

12. The crystal form of (S)-10,11-dihydro-10-hydroxy-5H-dibenz[b,f]azepine-5 carboxamide having the reference modification B, which Is characterised by a Powder X-ray diffraction diagram with d-spacings at 8.9, 7.8, 6.8, 6.3, 5.59, 4.13, 3.90, 3.69, 3.29, 2.60 Å and comprising less than 5% of reference modification A, which is characterised by a powder X-ray diffraction diagram with d-spacings at 12.6, 8.8, 7.5, 6.28, 5.24, 4.93, 3.84, 3.74, 3.42 Å.

13. A crystal modification of (S)-10,11-dihydro-10-hydroxy-5H dibenz[b,f]azepine-5-carboxamide having a melting point between 193.0 and 197.0° C.

14. A pharmaceutical composition which comprises a crystal form according to claim 6 together with a pharmaceutically acceptable carrier.

15. Method of treating a warm-blooded animal suffering from epilepsy by administering a dosage of 10,11-dihydro-10-hydroxy-5H-dibenz[b,f]azepine-5-carboxamide according to claim 6 which is effective for treating said disease to a warm blooded animal requiring such treatment.

16. (canceled)

17. (canceled)

18. A process for the preparation of (R)- or (S)-10,11-dihydro-10-hydroxy-5H-dibenz[b,f]azepine-5-carboxamide having reference modification B which is characterised by a powder X-ray diffraction diagram with d-spacings at 8.9, 7.8, 6.8, 6.3, 5.59, 4.13, 3.90, 3.69, 3.29, 2.60 Å, comprising the following steps,
(a) (R)- or (S)-10,11-dihydro-10-hydroxy-5H-dibenz[b,f]azepine-5-carboxamide are prepared according to a process according to any one of claim 1, and
(b) the obtained product having reference modification A which is characterised by a powder X-ray diffraction diagram with d-spacings at 12.6, 8.8, 7.5, 6.28, 5.24, 4.93, 3.84, 3.74, 3.42 Å or being in an amorphous form, is subjected to phase equilibration in a suitable solvent.

19. A process for the preparation of (R)- or (S)-10,11-dihydro-10-hydroxy-5H-dibenz[b,f]azepine-5-carboxamide having crystal form reference modification B which is characterised by a powder X-ray diffraction diagram with d-spacings at 8.9, 7.8, 6.8, 6.3, 5.59, 4.13, 3.90, 3.69, 3.29, 2.60 Å, comprising the following steps,
(a) (R)- or (S)-10,11-dihydro-10-hydroxy-5H-dibenz[b,f]azepine-5-carboxamide are prepared according to a process according to any one of claim 1, and
(b) the obtained product having reference modification A which is characterised by a powder X-ray diffraction diagram with d-spacings at 12.6, 8.8, 7.5, 6.28, 5.24, 4.93, 3.84, 3.74, 3.42 Å or being in an amorphous form, is dissolved in a suitable solvent and a crystal of (R)- or (S)-10,11-dihydro-10-hydroxy-5H-dibenz[b,f]azepine-5-carboxamide, respectively, having reference modification B is added.

20. A process for the preparation of (R)- or (S)-10,11-dihydro-10-hydroxy-5H-dibenz[b,f]azepine-5-carboxamide having reference modification B which is characterised by a Powder X-ray diffraction diagram with d-spacings at 8.9, 7.8, 6.8, 6.3, 5.59, 4.13, 3.90, 3.69, 3.29, 2.60 Å, wherein (R)- or (S)-10,11-dihydro-10-hydroxy-5H-dibenz[b,f]azepine-5-carboxamide having reference modification A which is characterised by a powder X-ray diffraction diagram with d-spacings at 12.6, 8.8, 7.5, 6.28, 5.24, 4.93, 3.84, 3.74, 3.42 Å or being in an amorphous form, is subjected to phase equilibration in a suitable solvent.

21. A process for the preparation of (R)- or (S)-10,11-dihydro-10-hydroxy-5H dibenz[b,f]azepine-5-carboxamide having reference modification B which is characterised by a powder X-ray diffraction diagram with d-spacings at 8.9, 7.8, 6.8, 6.3, 5.59, 4.13, 3.90, 3.69, 3.29, 2.60 Å, wherein (R)- or (S)-10,11-dihydro-10-hydroxy-5H-dibenz[b,f]azepine-5-carboxamide having reference modification A which is characterised by a powder X-ray diffraction diagram with d-spacings at 12.6, 8.8, 7.5, 6.28, 5.24, 4.93, 3.84, 3.74, 3.42 Å or being in an amorphous form, is dissolved in a suitable solvent and a crystal of (R)- or (S)-10,11-dihydro-10-hydroxy-5H-dibenz[b,f]azepine-5-carboxamide, respectively, having reference modification B is added.
